(19) United States
(12) Patent Application Publication
(10) Pub. No.: US 2026/0103706 A1
(43) Pub. Date: Apr. 16, 2026

(54) METHODS AND COMPOSITIONS FOR SINGLE-STRANDED RNA-INITIATED RISC ASSEMBLY

(71) Applicant: Ohio State Innovation Foundation, Columbus, OH (US)

(72) Inventor: Kotaro NAKANISHI, Columbus, OH (US)

(21) Appl. No.: 19/470,312

(22) PCT Filed: Apr. 1, 2024
(86) PCT No.: PCT/US2024/022510

§ 371 Date: Sep. 29, 2025

Related U.S. Application Data
(60) Provisional application No. 63/493,144, filed on Mar. 30, 2023.
(60) Provisional application No. 63/578,261, filed on Aug. 23, 2023.

(51) Int. Cl. C12N 15/113; C12N 15/10

(52) U.S. Cl. CPC C12N 15/113; C12N 15/10; C12N 2310/141; C12N 2330/30

(57) ABSTRACT

The present disclosure provides compositions comprising ss-guides with superior binding and degradation profiles, along with methods to design compositions comprising ss-guides with superior binding and degradation profiles.

### CROSS REFERENCE TO RELATED APPLICATIONS

This application claims priority to, and the benefit of, U.S. Provisional Patent Application No. 63/493,144, filed Mar. 30, 2023, entitled “METHODS AND COMPOSITIONS REGARDING OPTIMUM TARGET SEQUENCE OF SIRNAS FOR CLEAVAGE,” and U.S. Provisional Patent Application No. 63/578,261, filed Aug. 23, 2023, entitled “METHODS AND COMPOSITIONS REGARDING OPTIMUM TARGET SEQUENCE OF SIRNAS FOR CLEAVAGE,” which are incorporated by reference herein in their entireties.

### STATEMENT REGARDING FEDERALLY SPONSORED RESEARCH

This invention was made with Government Support under Grant No. GM138997 awarded by the National Institutes of Health. The Government has certain rights in the invention.

### REFERENCE TO SEQUENCE LISTING

The sequence listing submitted on Apr. 1, 2024, as an .XML file entitled “103361-477WO1_ST26” created on Mar. 28, 2024, and having a file size of 22,122 bytes is hereby incorporated by reference pursuant to 37 C.F.R. § 1.52(e)(5).

### FIELD

The present disclosure relates synthetic guide RNAs and RISC compositions, and methods thereof for regulating gene expression impacting a disease and/or disorder. BACKGROUND Four Argonaute proteins (AGO1-4) are expressed in humans. The four human Argonaute proteins are structurally very similar but nevertheless contain few non-conserved amino acids in their functional domains. Four conserved domains have been elucidated: the N-terminal domain (N), the PIWI/Argonaute/Zwille (PAZ) domain, the MID domain, and the P-element-induced whimpy tested (PIWI) domain. The PAZ domain is required for anchoring the 3′ end of guide RNAs.

MicroRNAs (miRNAs) and small interfering RNAs (siRNAs) are both loaded into AGOs efficiently as duplexes with the aid of seven chaperones. This duplex-initiated RISC assembly has been heavily studied and exploited in basic research and clinical therapeutics to silence the expression of target genes. Previous studies have reported that single-stranded (ss) RNA-derived guides (ss-guides) were incorporated into RNA-induced silencing complexes (RISCs). However, the molecular mechanisms and requirements for the single stranded (ss)-RNA-initiated RISC assembly have been poorly understood. What is needed in the art are ss-guide RNAs which are useful in the RISC assembly.

MiRNAs were defined to be about 22 nt because they are generated by Dicer, a molecular ruler. In contrast, there has been no clear definition of tinyRNAs (tyRNAs) to distinguish them from miRNAs and siRNAs.

### SUMMARY

The present disclosure provides synthetic nucleic acid compositions of tyRNAs comprising at least 14 nucleotides, fewer than 20 nucleotides, which is long enough to be captured at its 3′ end by a PAZ domain so that the AGO-tyRNA complex can complete a mature RISC (i.e., a functional RISC) (FIG. 2F). The present disclosure also provides RISC compositions comprising the synthetic nucleic acid compositions. The present disclosure provides methods of designing synthetic nucleic acid constructs for regulating gene expression impacted by a disease or disorder. The present disclosure also provides methods of regulating gene expression and/or detecting a variant AGO molecule to detecting, preventing, and/or treating a disease/disorder.

In one aspect, disclosed herein is a synthetic nucleic acid composition comprising a tyRNA, wherein the ss-guide tyRNA comprises at least 14 nucleotides and fewer than 20 nucleotides, and wherein the ss-guide RNA comprises a binding site for a PAZ domain required to trigger a conformational change from an immature RISC to a mature RISC (FIG. 2F).

In one aspect, disclosed herein is a synthetic RISC composition comprising the synthetic nucleic acid composition of any preceding aspect.

In some embodiments, the ss-guide RNA comprises 19 nucleotides. In some embodiments, the RISC is completely assembled or partially assembled. In some embodiments, the ss-guide RNA comprises 50%, 55%, 60%, 65%, 70%, 75%, 80%, 85%, 90%, 95%, or 100% complementarity to a binding region of a target nucleic acid, or any amount less than or in-between these values.

In some embodiments, the synthetic nucleic acid composition or the synthetic RISC composition further comprises a pharmaceutically acceptable carrier. In some embodiments, the pharmaceutically acceptable carrier comprises an excipient, diluent, filler, salt, buffer, stabilizer, solubilizer, lipid, stabilizer, or nanoparticle.

In one aspect disclosed herein is a method of designing a tyRNA to be used with a RISC assembly, the method comprising identifying a target nucleic acid, and sequencing together a plurality of nucleotides to generate a tyRNA, wherein the ss-guide RNA comprises at least 14 nucleotides and less than about 20 nucleotides.

In one aspect disclosed herein is a method of regulating expression of a target nucleic acid using an AGO molecule, the method comprising exposing the target nucleic acid to the AGO molecule loaded with a tyRNA comprising at least 14 nucleotides and less than about 20 nucleotides.

In some embodiments, the ss-guide RNA comprises 19 nucleotides. In some embodiments, the ss-guide RNA comprises a tyRNA, siRNA, shRNA, or a miRNA. In some embodiments, the ss-guide RNA comprises 50%, 55%, 60%, 65%, 70%, 75%, 80%, 85%, 90%, 95%, or 100% complementarity to a binding region of the target nucleic acid, or any amount less than or in-between these values. In some embodiments, the ss-guide RNA comprises a binding site for a PAZ domain of an RNA-induced silencing complex (RISC).

In some embodiments, the target nucleic acid comprises DNA or RNA. In some embodiments, the RNA comprises mRNA. In some embodiments, the RISC assembly comprises an AGO molecule. In some embodiments, the AGO comprises AGO1, AGO2, AGO3, or AGO4. In some embodiments, the target nucleic acid is silenced by the AGO.

In some embodiments, silencing comprises a gene-specific silencing. In some embodiments, the gene-specific silencing comprises a transcriptional gene silencing (TGS) activity or a post-transcriptional gene silencing (PTGS) activity. In some embodiments, said PTGS activity comprises RNA interference (RNAi) and/or translational attenuation.

In some embodiments, regulating expression of the target nucleic acid is used to treat a disease or disorder. In some embodiments, the disease or disorder is an infectious agent, a cancer, or a genetic defect.

In one aspect, disclosed herein is a method of detecting a variant AGO molecule, the method comprising a) exposing and binding an AGO molecule to a guide nucleic acid, wherein a 3′ region of the guide nucleic acid is bound within the wild-type AGO molecule, and wherein the 3′ region of the guide molecule is remains outside when bound to a variant AGO molecule (i.e., the F2L3 mutant), b) exposing the 3′ region of the guide nucleic acid to an exonuclease enzyme, wherein the exonuclease enzyme does not cleave the 3′ region of the guide nucleic acid when bound to the wild-type AGO molecule, and wherein the exonuclease enzyme cleaves the 3′ region of the guide nucleic acid when bound to the variant AGO molecule (i.e., the F2L3 mutant), and c) detecting the 3′ region of the guide nucleic acid; wherein the 3′ region is not detected when bound to the variant AGO molecule, and wherein the 3′ region is detected when bound to the control AGO molecule.

In some embodiments, the guide nucleic acid comprises a guide RNA. In some embodiments, the method further comprises loading the AGO molecule with a guide nucleic acid. In some embodiments, the guide RNA comprises a tinyRNA (tyRNA). In some embodiments, the guide RNA comprises 12-16 nucleotides in length. In some embodiments, the guide RNA is 14 nucleotides in length.

In some embodiments, the AGO molecule comprises AGO1, AGO2, AGO3, or AGO4. In some embodiments, the exonuclease enzyme comprises a 3′ to 5′ exonuclease enzyme. In some embodiments, the 3′ to 5′ exonuclease enzyme comprises ISG20 enzyme, or a variant thereof.

In some embodiments, the target nucleic acid comprises RNA or DNA. In some embodiments, the RNA comprises mRNA. In some embodiments, the method detects a disease or disorder. In some embodiments, the disease or disorder comprises an infectious agent, a cancer, or a genetic defect.

### BRIEF DESCRIPTION OF FIGURES

The accompanying figures, which are incorporated in and constitute a part of this specification, illustrate several aspects described below.

FIGS. 1A, 1B, 1C, 1D, 1E, and 1F show a functional PAZ domain and 14-nt or longer guide are essential for ssRNA-initiated RISC assembly. FIG. 1A shows the size-exclusion chromatography to test the interaction between ISG20 and AGO2/3 PAZ domain. FIG. 1B shows the schematics of the in vitro trimming assay of FLAG-AGO2-WT or -F2L3 programmed with a 5′-end radiolabeled 23-nt miR-20a. FIG. 1C shows a gel of the in vitro trimming assay using FLAG-AGO2-WT and FLAG-AGO2-F2L3 mutant. FIG. 1D shows the SDS-PAGE analysis of the purified proteins used in this study. FIG. 1E shows the in vitro trimming assay of FLAG-AGO2-WT programmed with a 5′-end radiolabeled 10, 14, 17, 18, 19, and 23-nt miR-20a. FIG. 1F shows the crystal structure of AGO2 in complex with a 21-nt guide RNA (PDB ID: 4W5N).

FIGS. 2A, 2B, 2C, 2D, 2E, and 2F show the mature RISC can capture the 3′ end of 19-nt or longer guide RNA. FIGS. 2A, 2B, 2C, and 2D show the time-course in vitro trimming assay of a 5′-end radiolabeled 23-nt miR-20a. Representative gel images of FLAG-AGO2-WT (FIG. 2A) and -AGO3-WT (FIG. 2B). Proportion of 13-23-nt miR-20a bound to AGO2 (FIG. 2C) and AGO3 (FIG. 2D). Mean SD. FIG. 2E shows the schematic of the guide 3′-end competition between the PAZ domain and ISG20. FIG. 2F shows the model of the ssRNA-initiated RISC assembly.

FIGS. 3A, 3B, 3C, 3D, 3E, 3F, 3G, 3H, and 3I show the results for the in vitro trimming assay. FIG. 3A shows the schematic of the competitive interactions of the 3′ end of guide RNA between the AGO PAZ domain and the processing enzymes. In the mature RISC conformation, both 5′ and 3′ ends of the guide RNA are recognized by the MID and PAZ domains. FIG. 3B shows the size-exclusion chromatography testing the interaction of ISG20 with the isolated AGO2 PAZ domain. FIG. 3C shows the SDS-PAGE analysis of the purified recombinant proteins used herein. The protein bands were visualized by Coomassie brilliant blue staining. FIG. 3D shows the definition of miRNAs and tyRNAs based on the guide length. Mature RISCs become tyRNA-associated RISCs (tyRISCs) upon the conversion of the miRNA to a tyRNA. FIG. 3E shows the schematic of the in vitro trimming assay of FLAG-AGO programmed with a 5′-end radiolabeled 23-nt miR-20a. FIG. 3F shows the time-course in vitro trimming assay of a 5′-end radiolabeled 23-nt miR-20a. Left: The relative abundance of 13-23-nt miR-20a bound to FLAG-AGO2 (top) and -AGO3 (bottom) over 8 hours was quantified from three independent experiments and shown with mean±SD. The guide lengths with the highest and second highest abundances at each time point are highlighted with red and orange asterisks, respectively. 10% relative abundance is depicted as dotted pink lines. Right: Representative gel images of the guide trimming on FLAG-AGO2 (top) and -AGO3 (bottom). FIG. 3G shows the time-course in vitro trimming assay of 5′-end radiolabeled 21-nt let-7a bound to FLAG-AGO3. Left: The relative abundance of 13-21-nt let-7a. Right: Representative gel images of the guide trimming. FIGS. 3H, 3G, and 3I show the time-course in vitro trimming assay of 5′-end radiolabeled 23-nt chimeric guides on FLAG-AGO2 (FIG. 3H) and -AGO3 (FIG. 3I). 500, 200, and 80 pmol of ISG20 were used for Chimeric-poly(U) (top), -poly(A) (middle), and -poly(C) (bottom), respectively.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction below, above, or in between the given ranges as compared to native or control levels.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

As used herein, “wild-type” refers to the genetic and physical characteristics of the typical form of a species as it occurs in nature. A wild-type or wild type characteristic is conceptualized as a product of the standard “normal” allele at a gene locus, in contrast to that produced by a non-standard “mutant” allele.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

“Prognosis” may refer to a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. Prognosis may also include prediction of favorable responses to cancer treatments, such as a conventional cancer therapy.

As used herein, the term “tissue sample” (the term “tissue” is used interchangeably with the term “tissue sample”) includes any material composed of one or more cells, either individual or in complex with any matrix obtained from a patient. The definition includes any biological or organic material and any cellular subportion, product or by-product thereof.

As used herein, the term “biological fluid” refers to a fluid containing cells and compounds of biological origin, and may include blood, stool or feces, lymph, urine, serum, pus, saliva, seminal fluid, tears, urine, bladder washings, colon washings, sputum or fluids from the respiratory, alimentary, circulatory, or other body systems. For the purposes of the present disclosure the “biological fluids”, the nucleic acids containing the biomarkers may be present in a circulating cell or may be present in cell-free circulating DNA or RNA.

“Expression” as used herein refers to the process by which information from a gene is used in the synthesis of a functional gene product that enables it to produce a peptide/protein end product, and ultimately affect a phenotype, as the final effect.

As used herein, the term “genetically modified” refers to a living cell, tissue, or organism whose genetic material has been altered using genetic engineering techniques. The genetic modification results in an alteration that does not occur naturally by mating and/or natural recombination. Modified genes can be transferred within the same species, across species (creating transgenic organisms), and across kingdoms. New, exogenous genes can be introduced, or endogenous genes can be enhanced, altered, or knocked out.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of disease or disorder), during early onset (e.g., upon initial signs and symptoms of disease or disorder), or after an established development of disease or disorder.

The term “interaction” refers to an action that occurs as two or more objects have an effect on one another either with or without physical contact. In terms of biological interactions, cell, proteins, and other macromolecules can have said effects on one another to impact biological functions, such as cell/tumor growth, cell death, and cell signaling pathways.

The term “detect” or “detecting” refers to an output signal released for the purpose of sensing of physical phenomenon. An event or change in environment is sensed and signal output released in the form of light, heat, color change, or the like.

A “nucleotide” is a compound consisting of a nucleoside, which consists of a nitrogenous base and a 5-carbon sugar, linked to a phosphate group forming the basic structural unit of nucleic acids, such as DNA or RNA. The four types of nucleotides are adenine (A), cytosine (C), guanine (G), and thymine (T), each of which are bound together by a phosphodiester bond to form a nucleic acid molecule.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes. A single-stranded oligonucleotide can exist as a linear molecule without any hydrogen-bonded nucleotides, or can fold three-dimensionally to form hydrogen bonds between individual nucleotides along the single stranded oligonucleotide.

The term “polynucleotide” refers to a single or double stranded polymer composed of nucleotide monomers. Polynucleotides can be any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “polynucleotide sequence” is the alphabetical representation of a polynucleotide molecule. In some embodiments, the polynucleotide is composed of nucleotide monomers of generally greater than 100 nucleotides in length and up to about 8,000 or more nucleotides in length.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

As used herein, “guide RNA” refer to a specifically designed RNA sequence that recognizes a target nucleic acid of interest and directs an enzyme, including but not limited to an exonuclease enzymes and RNA-induced silencing complex enzymes (such as, for example Argonaute protein (AGOs)) to the target nucleic acid for gene editing.

The term “mRNA” refers to messenger ribonucleic acid, or single stranded molecule of RNA that corresponds to the genetic sequence of a gene, and is translated by a ribosome in the process of synthesizing a protein. mRNA is created during the process of transcription, where a gene is converted into a primary transcript mRNA (or pre-mRNA). The primary transcript is further processed through RNA splicing to only contain regions that will encode protein. mRNA can also be targeted for epigenetic modifications, such as methylation, to impact mRNA translation, nuclear retention, nuclear export, processing, and splicing.

A nuclease is an enzyme capable of cleaving the phosphodiester bonds between nucleotides of nucleic acids. Nuclease can possess properties to cause double or single stranded breaks to target nucleic acids. Nucleases are commonly used in gene editing practices to modify a host genome to express or inhibit a target gene. An “exonuclease” refers to a type of enzyme essential to genome stability by acting to cleave, trim, or cut the free ends (such as the three prime (3′) end or the five prime (5′) end) of nucleic acids, including but not limited to DNA. Exonucleases are also involved in several aspects of cellular metabolism and maintenance.

As used herein, “RNAi” or RNA interference” refers to a process where small RNA molecules, including but not limited to tinyRNA, cityRNA, siRNA, miRNA, and shRNA, can shut down gene expression by binding and blocking the mRNA, protein translation enzymes, or a combination thereof, from performing intended functions.

“Downstream” means in a direction of transcription, the direction of transcription being from a promoter sequence to a RNA-encoding sequence. For a template strand of a double-stranded DNA molecule, the direction of transcription is 3′ to 5′. For a non-template strand of the double-stranded DNA molecule, the direction of transcription is 5′ to 3′. “Upstream” means in a direction opposite the direction of transcription. “Upstream” and “downstream” may be used in reference to either strand of a double-stranded DNA molecule even when relative to a sequence on one strand of a double-stranded DNA molecule.

The term “complementary” or “complementarity” refers to the topological compatibility or matching together of interacting surfaces of two molecules (e.g., a probe molecule and its target, particularly a DNA guide molecule and a target RNA molecule). Thus, the two molecules (e.g., target and its probe) can be described as complementary, and furthermore, the contact surface characteristics are complementary to each other. In the case of nucleotides or polynucleotides (e.g., DNA or RNA), the two molecules are complementary if they have sufficiently compatible nucleotide base-pairs such that the two molecules can hybridize. The term “complementary,” as it relates to nucleotide molecules (e.g., nucleotides, oligonucleotides, polynucleotides, modified nucleotides, etc.), is intended to include two or more nucleotide molecules which have 100% complementarity (e.g., each nucleotide in a sequence of one molecule is the nucleotide base-pair complement of an adjacent nucleotide in a sequence of the second molecule, in sequential order) as well as two or more nucleotide molecules which have less than 100% complementarity but which hybridize under the conditions of the methods disclosed herein.

The term “hybridization” or “hybridizes” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “recombinant” refers to a human manipulated nucleic acid (e.g. polynucleotide) or a copy or complement of a human manipulated nucleic acid (e.g. polynucleotide), or if in reference to a protein (i.e., a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a pre-sequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and awash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al. One of skill will recognize that these values can be appropriately adjusted to determine corresponding identity of proteins encoded by two nucleotide sequences by taking into account codon degeneracy, amino acid similarity, reading frame positioning and the like. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

### Synthetic Compositions

The present disclosure provides synthetic nucleic acid compositions comprising at least 14 nucleotides, fewer than 20 nucleotides, and a binding site for a PAZ domain of a RISC. The present disclosure also provides RISC compositions comprising the synthetic nucleic acid compositions.

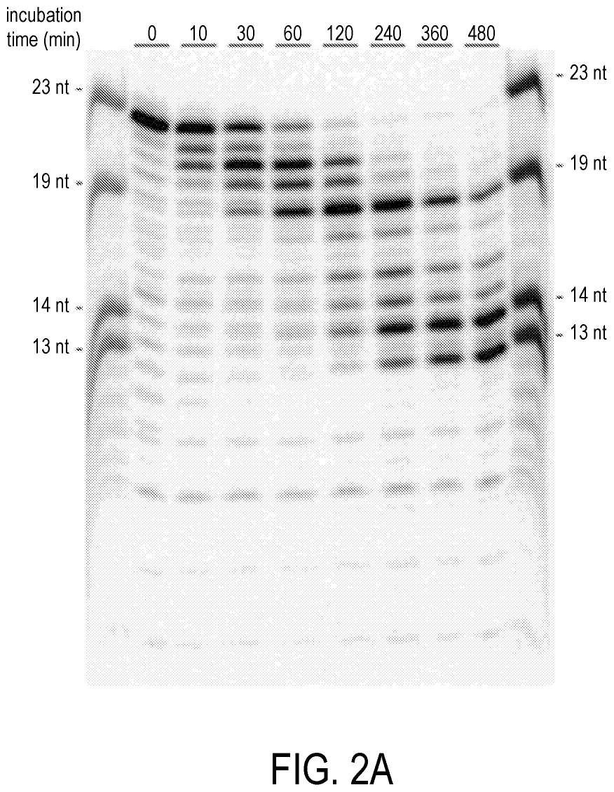

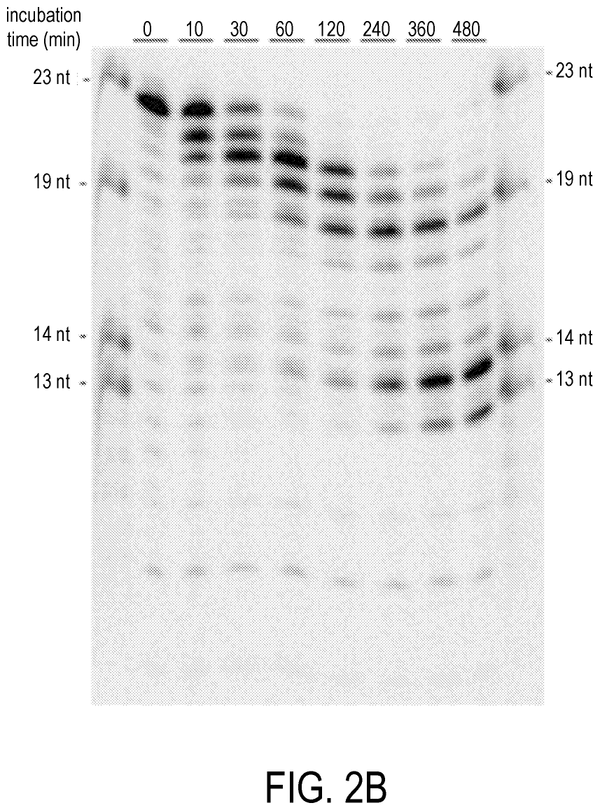

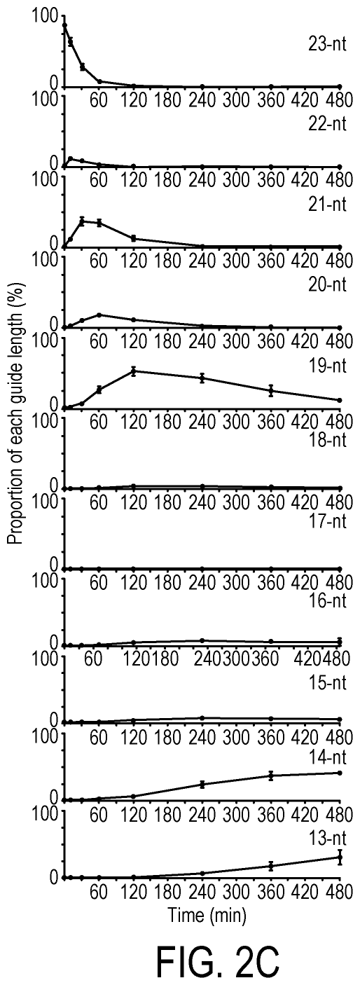

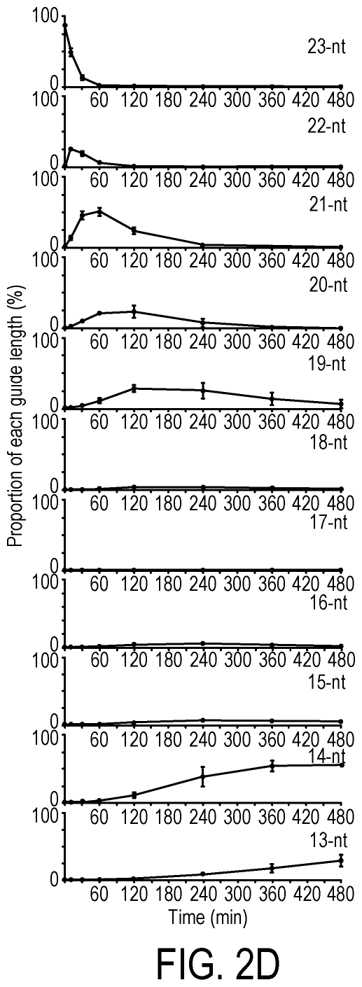

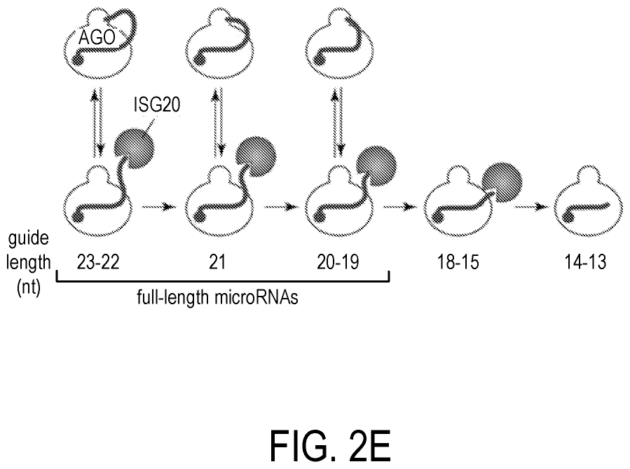

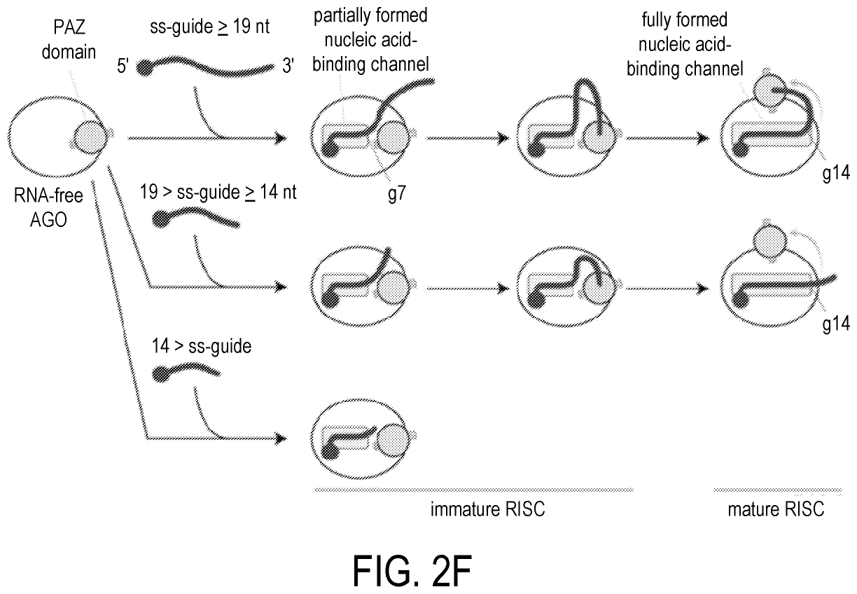

In one aspect, disclosed herein is a synthetic nucleic acid composition comprising a tyRNA, wherein the ss-guide RNA comprises at least 14 nucleotides and fewer than 20 nucleotides, and wherein the ss-guide RNA comprises a binding site for a PAZ domain to trigger a conformational change from immature RISC to mature RISC (FIG. 2F).

In one aspect, disclosed herein is a synthetic nucleic acid composition comprising a ss-guide DNA, wherein the ss-guide DNA comprises at least 14 nucleotides and fewer than 20 nucleotides, and wherein the ss-guide DNA comprises a binding site for a PAZ domain of an RNA-induced silencing complex (RISC).

In one aspect, disclosed herein is a synthetic RISC composition comprising the synthetic nucleic acid composition of any preceding aspect.

In some embodiments, the ss-guide RNA comprises 14, 15, 16, 17, 18, or 19 nucleotides. In some embodiments, the ss-guide RNA comprises 19 nucleotides. In some embodiments, the ss-guide DNA comprises 14, 15, 16, 17, 18, or 19 nucleotides. In some embodiments, the ss-guide DNA comprises 19 nucleotides. In some embodiments, the RISC is completely assembled or partially assembled. In some embodiments, the ss-guide RNA comprises 50%, 55%, 60%, 65%, 70%, 75%, 80%, 85%, 90%, 95%, or 100% complementarity to a binding region of a target nucleic acid, or any amount less than or in-between these values. In some embodiments, the ss-guide DNA comprises 50%, 55%, 60%, 65%, 70%, 75%, 80%, 85%, 90%, 95%, or 100% complementarity to a binding region of a target nucleic acid, or any amount less than or in-between these values.

In some embodiments, the synthetic nucleic acid composition or the synthetic RISC composition further comprises a pharmaceutically acceptable carrier. “Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

### Methods

The present disclosure provides methods of designing synthetic nucleic acid constructs for regulating gene expression impacted by a disease or disorder. The present disclosure also provides methods of regulating gene expression and/or detecting a variant AGO molecule to detecting, preventing, and/or treating a disease/disorder.

In one aspect disclosed herein is a method of designing a ss-guide RNA to be used with a RISC assembly, the method comprising identifying a target nucleic acid, and sequencing together a plurality of nucleotides to generate a ss-guide RNA, wherein the ss-guide RNA comprises at least 14 nucleotides and less than about 20 nucleotides.

In one aspect disclosed herein is a method of designing a ss-guide DNA to be used with a RISC assembly, the method comprising identifying a target nucleic acid, and sequencing together a plurality of nucleotides to generate a ss-guide DNA, wherein the ss-guide DNA comprises at least 14 nucleotides and less than about 20 nucleotides.

In one aspect disclosed herein is a method of regulating expression of a target nucleic acid using an AGO molecule, the method comprising exposing the target nucleic acid to the AGO molecule loaded with a ss-guide RNA comprising at least 14 nucleotides and less than about 20 nucleotides.

In one aspect disclosed herein is a method of regulating expression of a target nucleic acid using an AGO molecule, the method comprising exposing the target nucleic acid to the AGO molecule loaded with a ss-guide DNA comprising at least 14 nucleotides and less than about 20 nucleotides.

In some embodiments, the ss-guide RNA or the ss-guide DNA comprises 14, 15, 16, 17, 18, or 19 nucleotides. In some embodiments, the ss-guide RNA or the ss-guide DNA comprises 19 nucleotides. In some embodiments, the ss-guide RNA comprises a cityRNA, siRNA, shRNA, or a miRNA. As used herein, “siRNA” refers to short interfering RNA or silencing RNA that are a class of double stranded non-coding RNA molecules. Said siRNA molecule typically comprises between 20, 21, 22, 23, or 24 nucleotides. As used herein, “shRNA” refers to short hairpin RNA or small hairpin RNA is an artificial RNA molecule with a tight hairpin turn that is used to silence target gene expression. The turn within the artificial RNA molecule prevents or silence gene expression of the desired or target gene. As used herein, miRNA refers to small, single stranded, non-coding RNA molecules comprising between 19-25 nucleotides. In a specific example, the molecule is about 21, 22, or 23 nucleotides in length. miRNA molecules often resemble siRNA molecules, except miRNA molecules are derived from regions of RNA transcripts that fold back on themselves to form short hairpins, whereas siRNA molecules are derived from longer regions of double-stranded RNA. TinyRNAs (tyRNAs) are <17-nucleotide (nt) guide RNAs associated with Argonaute proteins (AGOs).

The present disclosure provides tyRNAs that can change the RISC conformation from an immature RISC to a mature RISC, thus in some embodiments, the ss-guide RNA, including but not limited to tyRNA (such as, for example a 14-nt ss-RNA), can change the RISC conformation from immature to mature.

In some embodiments, the ss-guide RNA or ss-guide DNA comprises 50%, 55%, 60%, 65%, 70%, 75%, 80%, 85%, 90%, 95%, or 100% complementarity to a binding region of the target nucleic acid, or any amount less than or in-between these values. In some embodiments, the ss-guide RNA or ss-guide DNA comprises a binding site for a PAZ domain of an RNA-induced silencing complex (RISC).

In some embodiments, the target nucleic acid comprises DNA or RNA. In some embodiments, the RNA comprises mRNA. In some embodiments, the RISC assembly comprises an AGO molecule. In some embodiments, the AGO comprises AGO1, AGO2, AGO3, or AGO4. In some embodiments, AGO 2 and AGO 3 retain slicer activity. In some embodiments, AGO1, AGO2, AGO3, and AGO4 recognizes the binding site of the target nucleic acid. In some embodiments, the target nucleic acid is silenced by AGO 2 or AGO 3.

Gene silencing refers to the regulation of gene expression in a cell to prevent expression of one or more genes. Gene silencing activity can occur at the level of gene transcription, protein translation, or a combination thereof. The phenomena of gene silencing has been harnessed and reengineered to produce therapeutics to combat diseases and disorders, including but not limited to cancer, infectious diseases, neurodegenerative diseases, and genetic disorders. It should be noted that gene silencing can be used interchangeably with the terms “gene knockdown”, “RNAi”, “gene-specific silencing”, “transcriptional gene silencing”, and “post-transcriptional gene silencing”.

In some embodiments, silencing comprises a gene-specific silencing. In some embodiments, the gene-specific silencing comprises a transcriptional gene silencing (TGS) activity or a post-transcriptional gene silencing (PTGS) activity. In some embodiments, said PTGS activity comprises RNA interference (RNAi) and/or translational attenuation. “Silencing” can mean a reduction in expression or activity by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

In some embodiments, regulating expression of the target nucleic acid is used to treat a disease or disorder. In some embodiments, the disease or disorder is an infectious agent, a cancer, or a genetic defect.

In one aspect, disclosed herein is a method of detecting a variant AGO molecule, the method comprising a) exposing and binding an AGO molecule to a guide nucleic acid, wherein a 3′ region of the guide nucleic acid is bound within the variant AGO molecule, and wherein the 3′ region of the target molecule is remains outside when bound to a control AGO molecule, b) exposing the 3′ region of the target nucleic acid to an exonuclease enzyme, wherein the exonuclease enzyme does not cleave the 3′ region of the guide nucleic acid when bound to the variant AGO molecule, and wherein the exonuclease enzyme cleaves the 3′ region of the guide nucleic acid when bound to the control AGO molecule, and c) detecting the 3′ region of the guide nucleic acid; wherein the 3′ region is not detected when bound to the variant AGO molecule, and wherein the 3′ region is detected when bound to the control AGO molecule.

As used herein, “variant”, “mutant”, or “derivative” refer to a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences-a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

In some embodiments, the guide nucleic acid comprises a guide RNA. In some embodiments, the method further comprises loading the AGO molecule with a target nucleic into the AGO molecule. In some embodiments, the guide RNA comprises a tyRNA. In some embodiments, the guide RNA or guide DNA comprises 12, 13, 14, 15, or 16 nucleotides in length. In some embodiments, the guide RNA or guide DNA is 14 nucleotides in length.

In some embodiments, the exonuclease enzyme comprises a 3′ to 5′ exonuclease enzyme.

In some embodiments, the 3′ to 5′ exonuclease enzyme comprises ISG20 enzyme, or a variant thereof.

In some embodiments, the target nucleic acid comprises RNA or DNA. In some embodiments, the RNA comprises mRNA.

In some embodiments, the method of any preceding aspect detects, treats, and/or prevents a disease or disorder. In some embodiments, the disease or disorder comprises an infectious agent, a cancer, or a genetic defect.

In some embodiments, the infectious agent comprises a virus, a bacteria, a fungus, or a parasite including, but not limited to Herpes Simplex virus- 1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, Human Immunodeficiency virus type-2, M. tuberculosis, M. bovis, M. bovis strain BCG, BCG substrains, M. avium, M. intracellular, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetii, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, other Yersinia species, Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, Alternaria alternata, Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, Entamoeba histolytica, or any combinations thereof.

In some embodiments, the infectious agent causes an infectious disease including, but not limited to common cold, influenza (including, but not limited to human, bovine, avian, porcine, and simian strains of influenza), measles, acquired immune deficiency syndrome/human immunodeficiency virus (AIDS/HIV), anthrax, botulism, cholera, campylobacter infections, chickenpox, chlamydia infections, cryptosporidosis, dengue fever, diphtheria, hemorrhagic fevers, Escherichia coli (E. coli) infections, ehrlichiosis, gonorrhea, hand-foot-mouth disease, hepatitis A, hepatitis B, hepatitis C, legionellosis, leprosy, leptospirosis, listeriosis, malaria, meningitis, meningococcal disease, mumps, pertussis, polio, pneumococcal disease, paralytic shellfish poisoning, rabies, rocky mountain spotted fever, rubella, salmonella, shigellosis, small pox, syphilis, tetanus, trichinosis (trichinellosis), tuberculosis (TB), typhoid fever, typhus, west nile virus, yellow fever, yersiniosis, and zika.

In some embodiments, the cancer includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: ISG20 is a Good Tool to Trim AGO-Associated Guide RNAs

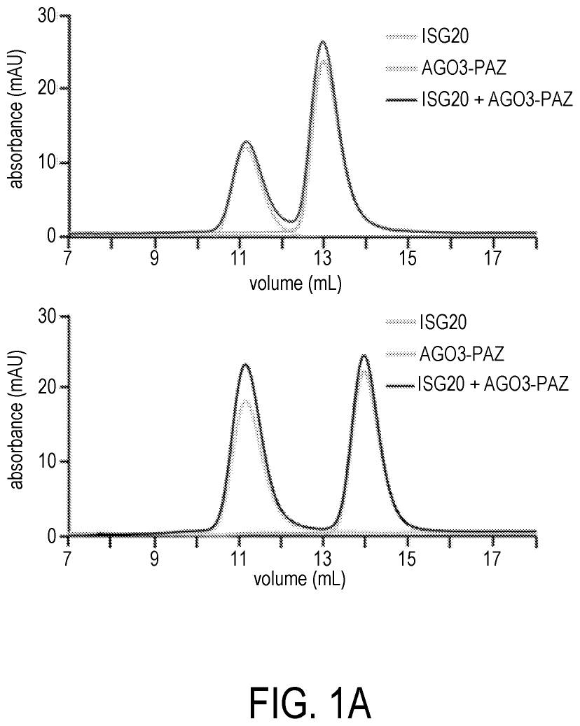

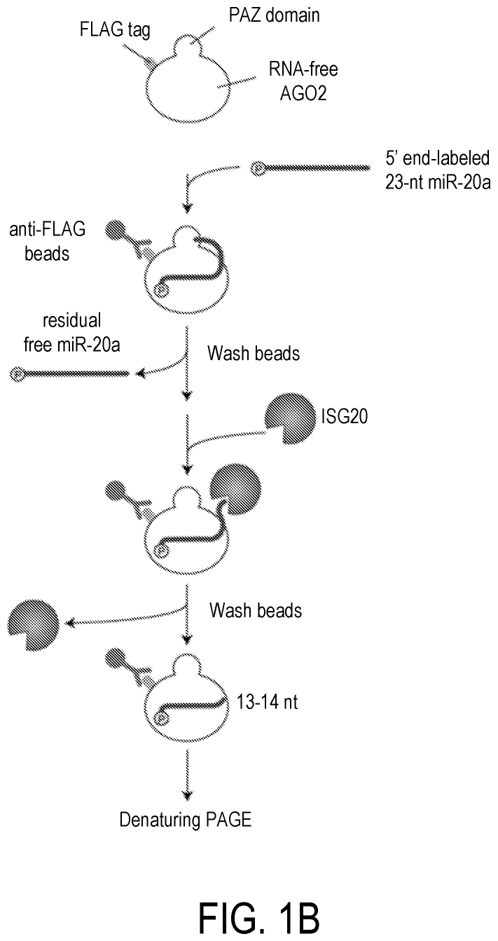

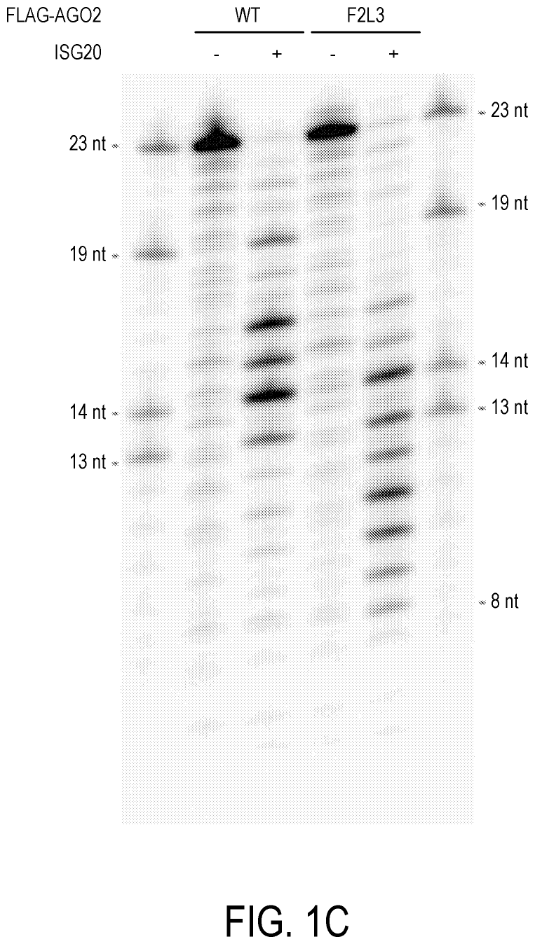

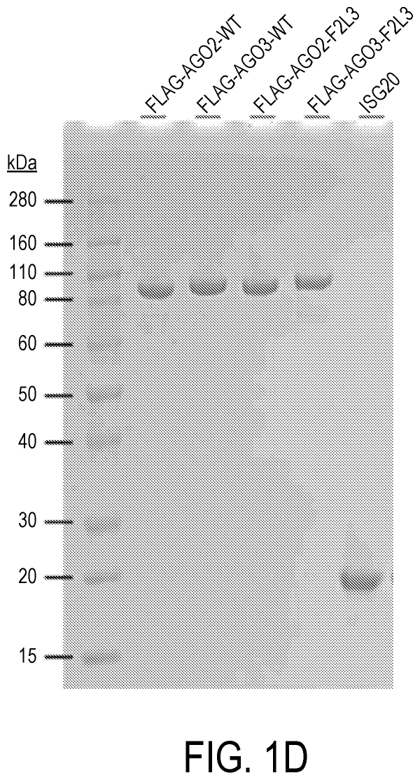

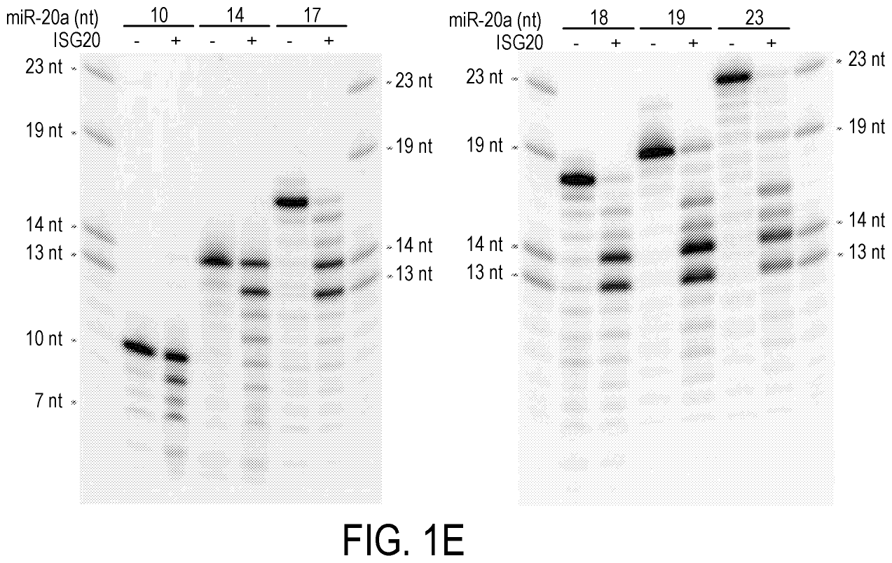

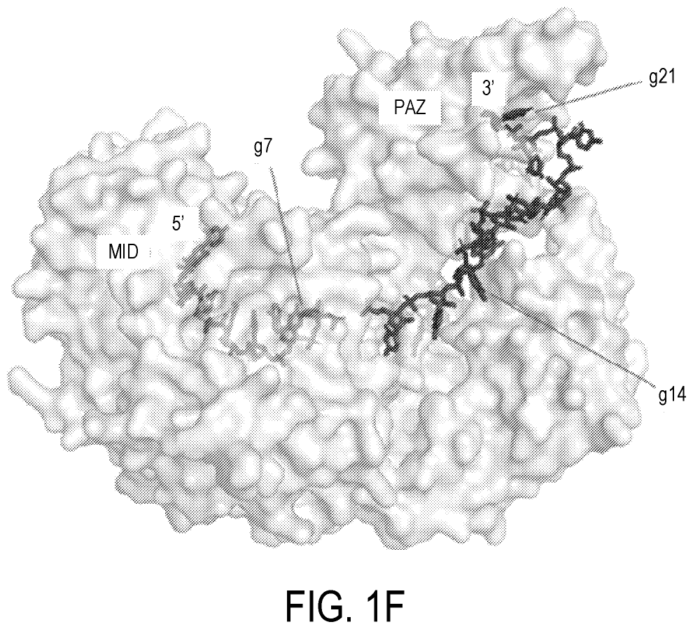

It was previously shown that after either duplex- or ssRNA-initiated RISC assembly, interferon-stimulated gene 20 kDa (ISG20), a 3′→5′ exonuclease, trimmed human AGO-associated guide RNAs and made them 13-14 nucleotides because the correctly assembled RISC forms a nucleic acid-binding channel that sequesters the g1-g13 (or -g14) (G. Sim et al., Manganese-dependent microRNA trimming by 3′-->5′ exonucleases generates 14-nucleotide or shorter tinyRNAs. Proc Natl Acad Sci US A 119, e2214335119 (2022). Therefore, the stop of ISG20-dependent guide trimming at g13 or g14 indicates that the AGO and guide completed their RISC assembly (i.e., forming mature RISC). Unlike small RNA degrading nuclease 1 (SDN1), whose 3′→5′ guide trimming is initiated by binding to the AGO PAZ domain, ISG20 has no interaction with the PAZ domain of AGO2 or AGO3 (FIG. 1A). Since ISG20 does not interfere with the PAZ domain's performance, this exonuclease was employed as a tool to investigate ssRNA-initiated RISC assembly.

METHODS: A Superdex 200 increase 10/300 GL column (GE Healthcare) was equilibrated with running buffer (150 mM NaCl, 20 mM Tris-HCl pH 7.5, 2 mM DTT). 4 nmol of purified AGO2 PAZ domain, SUMO-fused ISG20, or the mixture of both was injected to the column (GE Healthcare). The mixture was incubated on ice for 30 min prior to injection.

### Example 2: In Vitro Trimming Assay Using FLAG-AGO2 F2L3

After incubation with a 5′-end-radiolabeled 23-nt miR-20a, FLAG-tagged AGO2 wild-type (FLAG-AGO2-WT) was immobilized on anti-FLAG beads (FIG. 1). Any residual free guide was washed out thoroughly, and ISG20 was added to trim the AGO-associated guide. ISG20 trimmed the 23-nt miR-20a until the length became 13-14 nucleotides (FIGS. 1C and 1D), indicating that AGO2-WT and 23-nt ss-guides can assemble into a RISC. When the same experiment was repeated with an AGO2 mutant, whose PAZ domain fails to capture the guide 3′ end (FLAG-AGO2-F2L3), ISG20 continued to trim the guide down to 8 nucleotides (FIGS. 1C and 1D), indicating that AGO2-F2L3 and 23-nucleotide ss-guides partially form the nucleic acid-binding channel, which protects the g1-g8 but exposes the g9-g23 to the solvent. In addition, this result demonstrates that a functional PAZ domain capable of capturing the guide 3′ end is indispensable to the ssRNA-initiated RISC assembly.

METHODS: For the RISC assembly, 20 M recombinant FLAG-AGO2 or FLAG-AGO2 F2L3 was incubated for 1 hour at 37° C. with 2 M 5′ end-labeled 23-nt miR-20a in 1× Trimming Buffer (25 mM HEPES-KOH pH 7.5, 50 mM KCl, 5 mM DTT, 0.2 mM EDTA, 0.05 mg/mL BSA) with a total volume of 30 μL. The 30 μL assembled RISC reaction was pulled down by 150 L of anti-FLAG M2 beads (Sigma Aldrich), which were pre-washed with 1×PBS twice and 1× Trimming buffer twice, for 2 hours at RT. Then, the beads were washed with IP wash buffer (300 mM NaCl, 50 mM Tris-HCl pH 7.5, 5 mM MgCl₂, and 0.05% NP-40) 8 times. Next, the residual buffer was removed from the beads and they were washed with 1x Trimming buffer including 5 mM MnCl₂ twice. The 150 μL reaction solution was aliquoted into 50 μL and incubated with 500 pmol of ISG20 for 4 hours at 37° C. for the guide RNA trimming. After incubation with ISG20, 50 μL of the beads were moved to a new tube and washed with IP wash buffer 4 times, mixed with 2× urea quenching dye (8 M urea, 1 mM EDTA, 0.05% (w/v) xylene cyanol, 0.05% (w/v) bromophenol blue, 10% (v/v) phenol) and resolved on an 8 M urea 16% (w/v) polyacrylamide gel. Images were analyzed by the Typhoon Imaging System (GE Healthcare), quantified by Image Lab (Bio-Rad), and statistically analyzed using GraphPad Prism.

### Example 3: In Vitro Trimming Assay Using Different Lengths of miR-20a

When programmed with 17, 18, 19, or 23-nt miR-20a, FLAG-AGO2-WT protected their g1-g13 (or -g14) from trimming by ISG20 (FIG. 1E). Notably, 14-nt miR-20a also remained 13-14 nucleotides, albeit a small amount of unstoppable trimming which generated shorter guides (FIG. 1E). In contrast, the FLAG-AGO2-WT-associated 10-nt miR-20a was trimmed down to 7 nucleotides (FIG. 1E), forming an incomplete nucleic acid-binding channel, as seen in FLAG-AGO2-F2L3 (FIG. 1C). These results show that AGO2-WT and 14-nucleotide or longer ss-guides can assemble a RISC correctly (i.e., forming mature RISC in FIG. 2F) because the PAZ domain can capture the guide 3′ end. However, all the previously reported crystal structures of human RISCs show the hallmark architecture, where the PAZ domain cannot reach the 3′ end of 14-nucleotide guides (FIG. 1F). To determine the minimum guide length required for capture at its 3′ end by the PAZ domain of a mature RISC, a time-course in vitro trimming assay was performed using FLAG-AGO2-WT loaded with a 5′-end-radiolabeled 23-nt miR-20a. While the guide was trimmed to 22, 21, 20, 19, 14, and 13 nucleotides, no 15-18-nucleotide guide stably existed during guide trimming (FIGS. 2A and 2C). A similar trimming pattern was seen for FLAG-AGO3-WT (FIGS. 2B and 2D). These results indicate that when the guide remains 19 nucleotides or longer, the 3′ end is accessible to either the PAZ domain or ISG20. Still, once the guide becomes 18-nucleotides or shorter, the 3′ end is no longer captured by the PAZ domain and instead recognized by only ISG20 (FIG. 2E). Therefore, 19 nucleotides is the minimum guide length needed to be recognized by the PAZ domain in the context of mature RISC, which is consistent with the structural observation that the g14 is not accessible to the RISC PAZ domain (FIG. 1F).

These results support a mechanism of ssRNA-initiated RISC assembly (FIG. 2F). 14-nt ss guide RNAs can be loaded into AGO, recognized at its 3′ end by the PAZ domain, and capable of changing the conformation from the immature RISC to the mature RISC (FIG. 1E). However, the crystal structure shows that 14-nt guide RNA is not long enough to have its 3′ end reach the PAZ domain (FIG. 1F). This could be explained by a model shown in FIG. 2F. When a 14-nt guide RNA is loaded into AGO, the 3′ end must be located close to the PAZ domain. The binding of the 3′ to the PAZ domain triggers a conformational change from the immature RISC to the mature RISC, which is accompanied by the relocation of the PAZ domain (FIG. 2F).

METHODS: For the RISC assembly, 20 M recombinant FLAG-AGO2 was incubated for 1 hour at 37° C. with 2 M 5′ end-labeled 23-, 19-, 18-, 17-, 14-, or 10-nt miR-20a in 1× Trimming Buffer (25 mM HEPES-KOH pH 7.5, 50 mM KCl, 5 mM DTT, 0.2 mM EDTA, 0.05 mg/mL BSA) with a total volume of 30 μL. The 30 μL assembled RISC reaction was pulled down by 150 L of anti-FLAG M2 beads (Sigma Aldrich), which were pre-washed with 1×PBS twice and 1× Trimming buffer twice, for 2 hours at RT. Then, the beads were washed with IP wash buffer (300 mM NaCl, 50 mM Tris-HCl pH 7.5, 5 mM MgCl₂, and 0.05% NP-40) 8 times. Next, the residual buffer was removed from the beads and they were washed with 1× Trimming buffer including 5 mM MnCl₂ twice. The 150 μL reaction solution was aliquoted into 50 μL and incubated with 500 pmol of ISG20 for 4 hours at 37° C. for the guide RNA trimming. After incubation with ISG20, 50 L of the beads were moved to a new tube and washed with IP wash buffer 4 times, mixed with 2× urea quenching dye (8 M urea, 1 mM EDTA, 0.05% (w/v) xylene cyanol, 0.05% (w/v) bromophenol blue, 10% (v/v) phenol) and resolved on an 8 M urea 16% (w/v) polyacrylamide gel. Images were analyzed by the Typhoon Imaging System (GE Healthcare), quantified by Image Lab (Bio-Rad), and statistically analyzed using GraphPad Prism.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

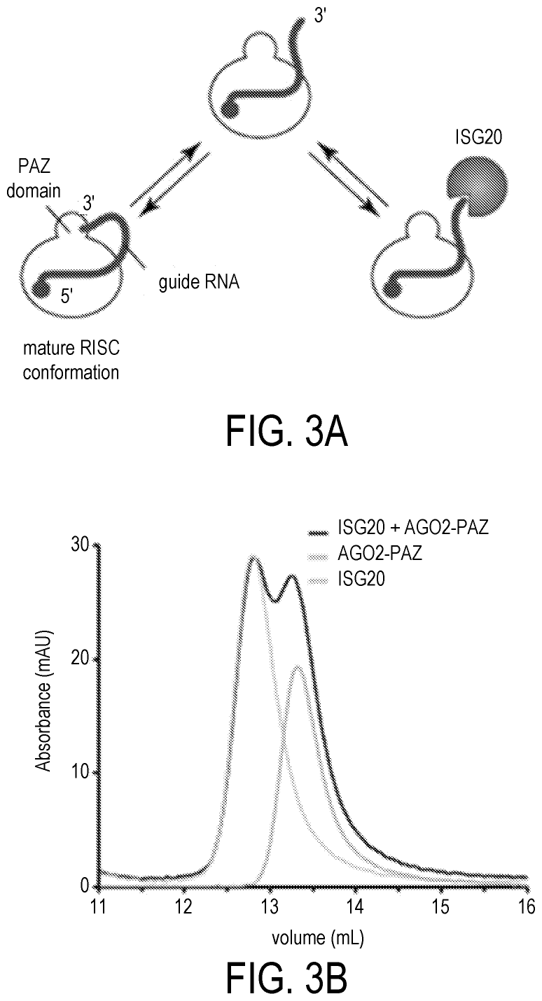

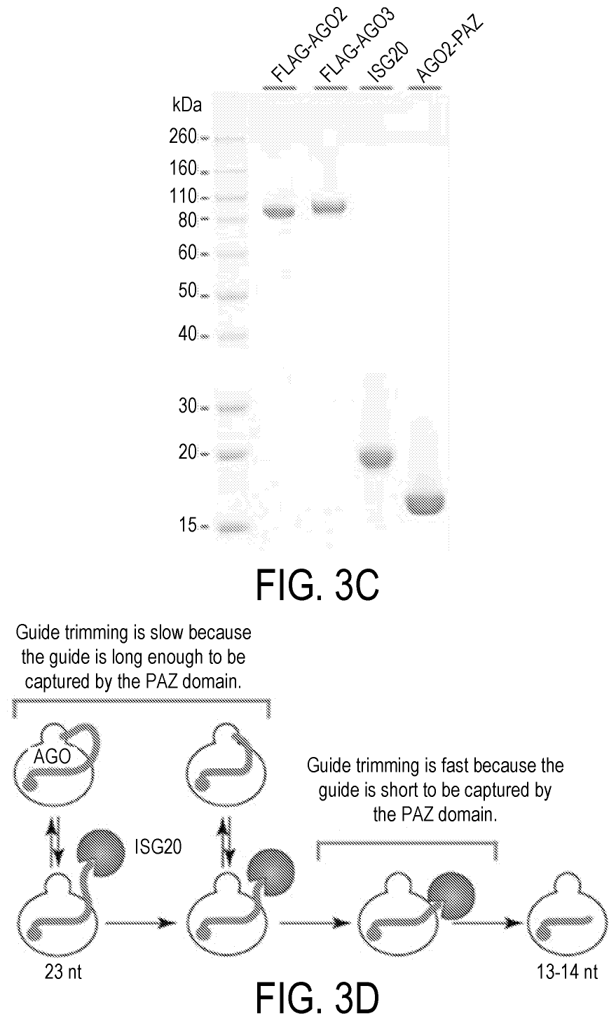

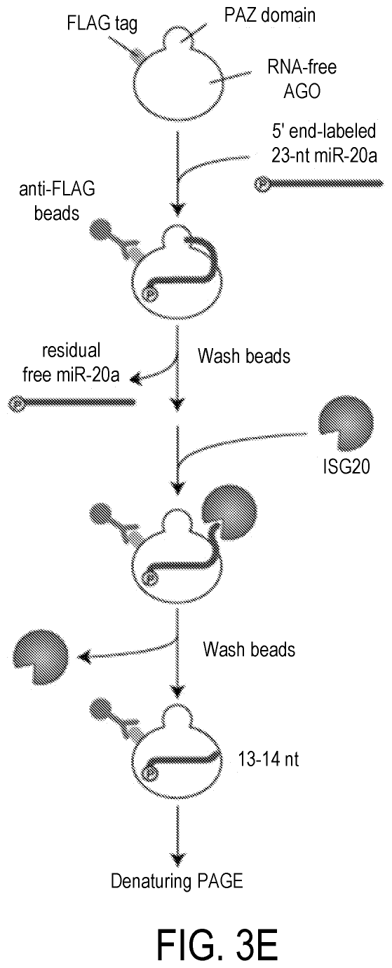

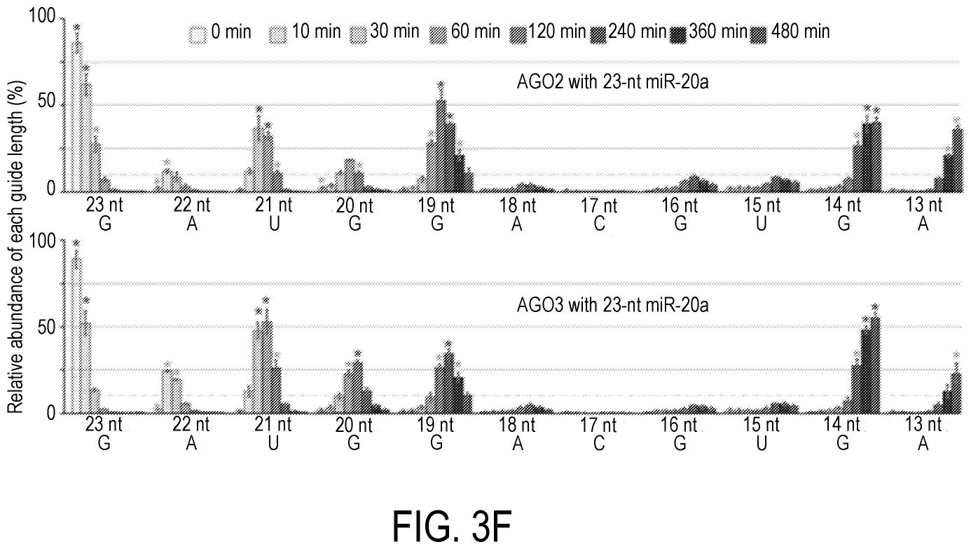

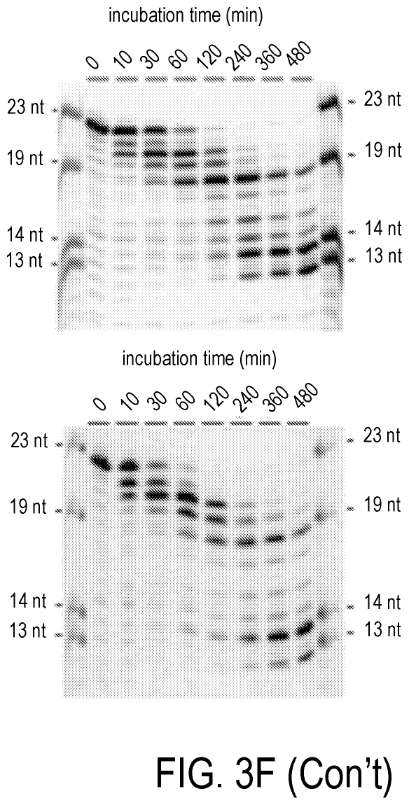

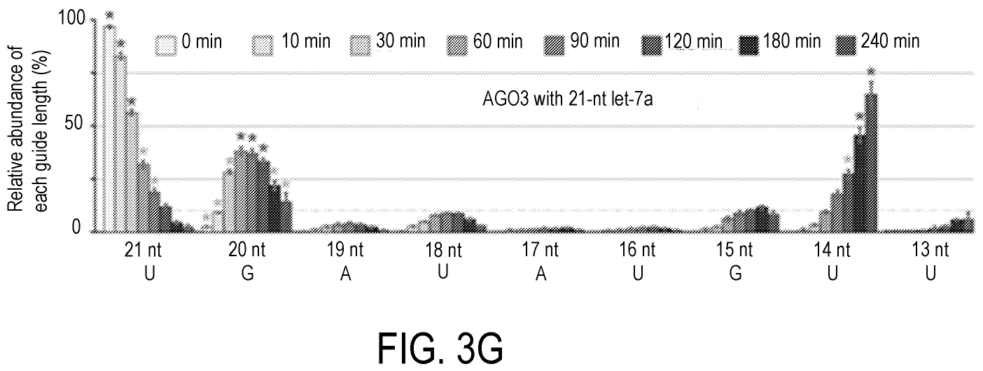

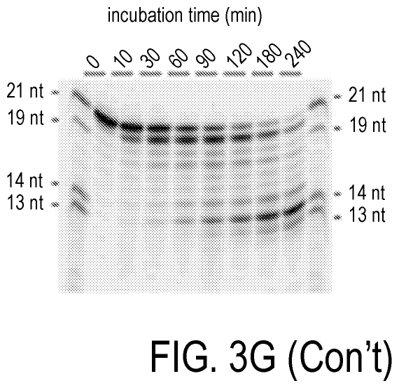

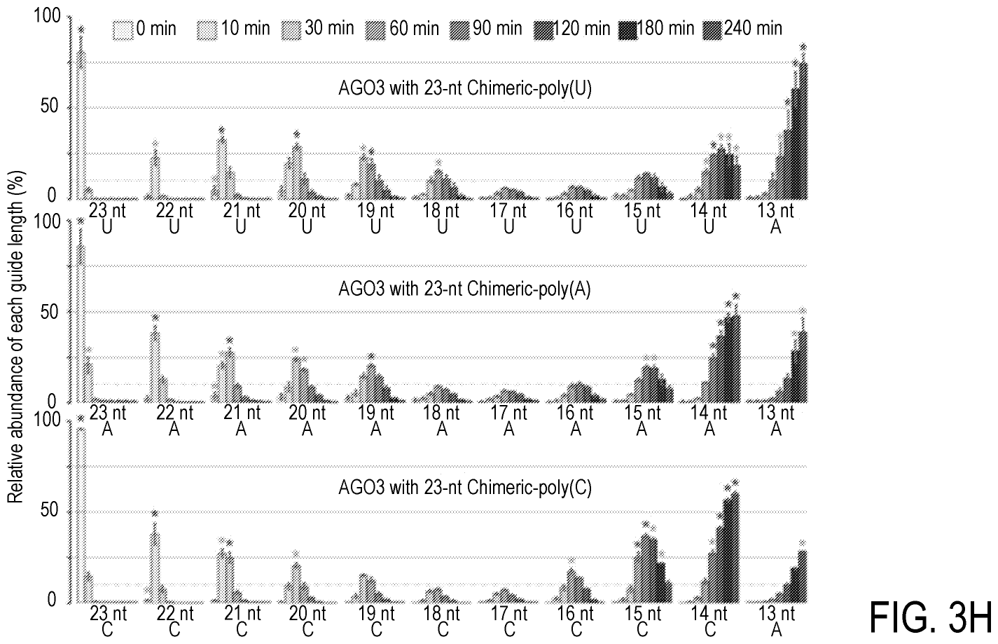

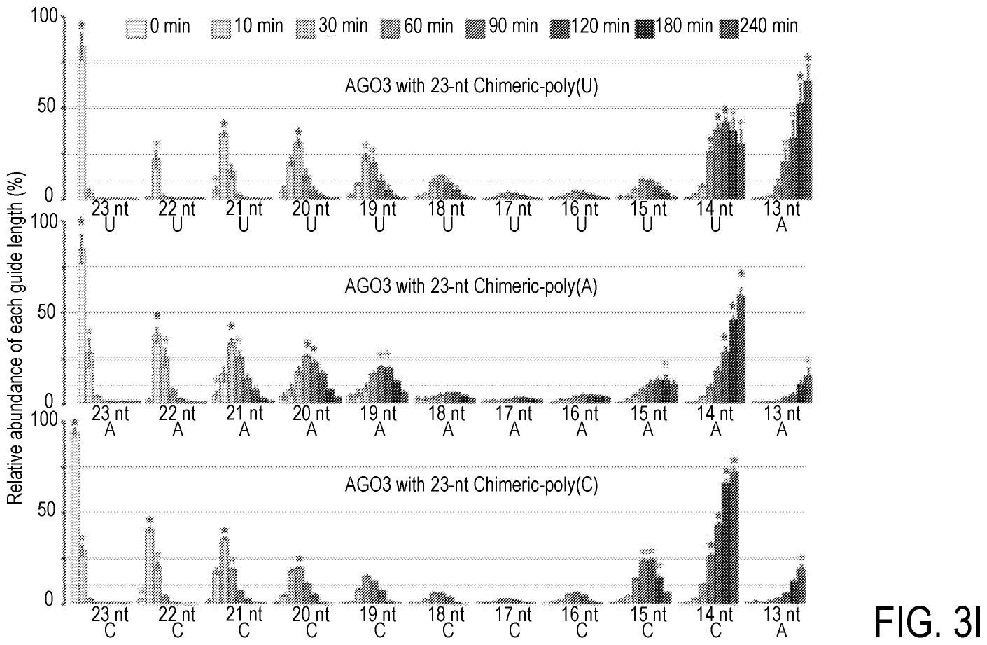

TABLE 1
Guide RNA sequences used in FIGS. 3G, 3H, and 3I.

| Guide RNAs | Sequence | |
|---|---|---|
| | 1 5 10 15 20 | |
| | \| \| \| \| \| | |
| 23-nt miR-20a | 5′-pUAAAGUGCUUAUAGUGCAGGUAG-3′ | SEQ ID NO: 10 |
| 21-nt let-7a | 5′-pUGAGGUAGUAGGUUGUAUAGU-3′ | SEQ ID NO: 11 |
| 23-nt chimeric poly(U) | 5′-pUAAAGUGCUUAUAUUUUUUUUUU-3′ | SEQ ID NO: 12 |
| 23-nt chimeric poly(A) | 5′-pUAAAGUGCUUAUAAAAAAAAAAA-3′ | SEQ ID NO: 13 |
| 23-nt chimeric poly(C) | 5′-pUAAAGUGCUUAUACCCCCCCCCC-3′ | SEQ ID NO: 14 |

SEQUENCES
1. SEQ ID NO: 1 - 14-nt miR-20a variant
UAAAGUGCUUAUAG
2. SEQ ID NO: 2 - 15-nt miR-20a variant
UAAAGUGCUUAUAGU
3. SEQ ID NO: 3 - 16-nt miR-20a variant
UAAAGUGCUUAUAGUG
4. SEQ ID NO: 4 - 17-nt miR-20a variant
UAAAGUGCUUAUAGUGC
5. SEQ ID NO: 5 - 18-nt miR-20a variant
UAAAGUGCUUAUAGUGCA
6. SEQ ID NO: 6 - 19-nt miR-20a variant
UAAAGUGCUUAUAGUGCAG
7. SEQ ID NO: 7 - Human AGO1 PAZ (A225-S369)
AQPVIEFMCEVLDIRNIDEQPKPLTDSQRVRFTKEIKGLKVEVTHCGQMKRKYRVCNVT
RRPASHQTFPLQLESGQTVECTVAQYFKQKYNLQLKYPHLPCLQVGQEQKHTYLPLEV
CNIVAGQRCIKKLTDNQTSTMIKATARS
8. SEQ ID NO: 8 - Human AGO2 PAZ (A227-R351)
AQPVIEFVCEVLDFKSIEEQQKPLTDSQRVKFTKEIKGLKVEITHCGQMKRKYRVCNVT
RRPASHQTFPLQQESGQTVECTVAQYFKDRHKLVLRYPHLPCLQVGQEQKHTYLPLEV
CNIVAGQR
9. SEQ ID NO: 9 - Human AGO3 PAZ (A228-R352)
AQPVIQFMCEVLDIHNIDEQPRPLTDSHRVKFTKEIKGLKVEVTHCGTMRRKYRVCNVT
RRPASHQTFPLQLENGQTVERTVAQYFREKYTLQLKYPHLPCLQVGQEQKHTYLPLEV
CNIVAGQR
10. SEQ ID NO: 10 - 23-nt miR-20a
pUAAAGUGCUUAUAGUGCAGGUAG
11. SEQ ID NO: 11 - 21-nt let-7a
pUGAGGUAGUAGGUUGUAUAGU
12. SEQ ID NO: 12 - 23-nt chimeric poly(U)
pUAAAGUGCUUAUAUUUUUUUUUU
13. SEQ ID NO: 13 - 23-nt chimeric poly(A)
pUAAAGUGCUUAUAAAAAAAAAAA
14. SEQ ID NO: 14 - 23-nt chimeric poly(C)
pUAAAGUGCUUAUACCCCCCCCCC

1. A synthetic nucleic acid composition comprising a ss-guide RNA, wherein the ss-guide RNA comprises at least 14 nucleotides and fewer than 20 nucleotides, and wherein the ss-guide RNA comprises a binding site for a PAZ domain of an RNA-induced silencing complex (RISC).

2. The synthetic nucleic acid composition of claim 1, wherein the ss-guide RNA comprises 19 nucleotides.

3. The synthetic nucleic acid composition of claim 1, wherein the RISC is completely assembled or partially assembled.

4. The synthetic nucleic acid composition of claim 1, wherein the ss-guide RNA comprises 50%, 55%, 60%, 65%, 70%, 75%, 80%, 85%, 90%, 95%, or 100% complementarity to a binding region of a target nucleic acid, or any amount less than or in-between these values.

5. The synthetic nucleic acid composition of claim 1, wherein the synthetic nucleic acid composition further comprises a pharmaceutically acceptable carrier.

6. The synthetic nucleic acid composition of claim 5 wherein the pharmaceutically acceptable carrier comprises an excipient, diluent, filler, salt, buffer, stabilizer, solubilizer, lipid, stabilizer, or nanoparticle.

7. A synthetic RISC composition comprising the synthetic nucleic acid composition of claim 1.

8. The synthetic RISC composition of claim 7, wherein the synthetic RISC composition further comprises a pharmaceutically acceptable carrier.

9. The synthetic RISC composition of claim 8, wherein the pharmaceutically acceptable carrier comprises an excipient, diluent, filler, salt, buffer, stabilizer, solubilizer, lipid, stabilizer, or nanoparticle.

10. A method of designing a guide RNA to be used with a RISC assembly, the method comprising:
a) identifying a target nucleic acid, and
b) sequencing together a plurality of nucleotides to generate a single stranded (ss)-guide RNA, wherein the ss-guide RNA comprises at least 14 nucleotides and less than about 20 nucleotides.

11. The method of claim 10, wherein the ss-guide RNA comprises 19 nucleotides.

12. The method of claim 10-e-11-, wherein the ss-guide RNA comprises a tinyRNA(tyRNA), siRNA, shRNA, or a miRNA.

13. The method of claim 10, wherein the ss-guide RNA comprises 50%, 55%, 60%, 65%, 70%, 75%, 80%, 85%, 90%, 95%, or 100% complementarity to a binding region of the target nucleic acid, or any amount less than or in-between these values.

14. The method of claim 10, wherein the ss-guide RNA comprises a binding site for a PAZ domain of an RNA-induced silencing complex (RISC).

15. The method of claim 10, wherein the target nucleic acid comprises DNA or RNA.

16. The method of claim 15, wherein the RNA comprises mRNA.

17. The method of claim 10, wherein the RISC assembly comprises an AGO molecule.

18. The method of claim 17, wherein the AGO comprises AGO1, AGO2, AGO3, or AGO4.

19. A method of regulating expression of a target nucleic acid using an AGO molecule, the method comprising exposing the target nucleic acid to the AGO molecule loaded with a guide RNA comprising at least 14 nucleotides and less than about 20 nucleotides.

20. The method of claim 19, wherein the target nucleic acid is silenced by the AGO.

21-45. (canceled)


SEQUENCE LISTING (14 sequences)

SEQ ID NO: 1 - RNA, 14 nt, synthetic construct
  misc_feature 1..14: note = Description of sequence: 14-nt miR-20a variant
  source 1..14: mol_type = other RNA
taaagtgcttatag

SEQ ID NO: 2 - RNA, 15 nt, synthetic construct
  misc_feature 1..15: note = Description of sequence: 15-nt miR-20a variant
  source 1..15: mol_type = other RNA
taaagtgcttatagt

SEQ ID NO: 3 - RNA, 16 nt, synthetic construct
  misc_feature 1..16: note = Description of sequence: 16-nt miR-20a variant
  source 1..16: mol_type = other RNA
taaagtgcttatagtg

SEQ ID NO: 4 - RNA, 17 nt, synthetic construct
  misc_feature 1..17: note = Description of sequence: 17-nt miR-20a variant
  source 1..17: mol_type = other RNA
taaagtgcttatagtgc

SEQ ID NO: 5 - RNA, 18 nt, synthetic construct
  misc_feature 1..18: note = Description of sequence: 18-nt miR-20a variant
  source 1..18: mol_type = other RNA
taaagtgcttatagtgca

SEQ ID NO: 6 - RNA, 19 nt, synthetic construct
  misc_feature 1..19: note = Description of sequence: 19-nt miR-20a variant
  source 1..19: mol_type = other RNA
taaagtgcttatagtgcag

SEQ ID NO: 7 - protein, 145 aa, Homo sapiens
  REGION 1..145: note = Description of sequence: Human AGO1 PAZ (A225-S369)
AQPVIEFMCEVLDIRNIDEQPKPLTDSQRVRFTKEIKGLKVEVTHCGQMKRKYRVCNVTRRPASHQTFPLQLESGQTVECTVAQYFKQKYNLQLKYPHLPCLQVGQEQKHTYLPLEVCNIVAGQRCIKKLTDNQTSTMIKATARS

SEQ ID NO: 8 - protein, 125 aa, Homo sapiens
  REGION 1..125: note = Description of sequence: Human AGO2 PAZ (A227-R351)
AQPVIEFVCEVLDFKSIEEQQKPLTDSQRVKFTKEIKGLKVEITHCGQMKRKYRVCNVTRRPASHQTFPLQQESGQTVECTVAQYFKDRHKLVLRYPHLPCLQVGQEQKHTYLPLEVCNIVAGQR

SEQ ID NO: 9 - protein, 125 aa, Homo sapiens
  REGION 1..125: note = Description of sequence: Human AGO3 PAZ (A228-R352)
AQPVIQFMCEVLDIHNIDEQPRPLTDSHRVKFTKEIKGLKVEVTHCGTMRRKYRVCNVTRRPASHQTFPLQLENGQTVERTVAQYFREKYTLQLKYPHLPCLQVGQEQKHTYLPLEVCNIVAGQR

SEQ ID NO: 10 - RNA, 23 nt, synthetic construct
  misc_feature 1..23: note = Description of sequence: 23-nt miR-20a
  source 1..23: mol_type = other RNA
  modified_base 1: mod_base = OTHER; note = monophosphate group attached
taaagtgcttatagtgcaggtag

SEQ ID NO: 11 - RNA, 21 nt, synthetic construct
  misc_feature 1..21: note = Description of sequence: 23-nt miR-20a
  source 1..21: mol_type = other RNA
  modified_base 1: mod_base = OTHER; note = monophosphate group attached
tgaggtagtaggttgtatagt

SEQ ID NO: 12 - RNA, 23 nt, synthetic construct
  misc_feature 1..23: note = Description of sequence: 23-nt miR-20a
  source 1..23: mol_type = other RNA
  modified_base 1: mod_base = OTHER; note = monophosphate group attached
taaagtgcttatatttttttttt

SEQ ID NO: 13 - RNA, 23 nt, synthetic construct
  misc_feature 1..23: note = Description of sequence: 23-nt miR-20a
  source 1..23: mol_type = other RNA
  modified_base 1: mod_base = OTHER; note = monophosphate group attached
taaagtgcttataaaaaaaaaaa

SEQ ID NO: 14 - RNA, 23 nt, synthetic construct
  misc_feature 1..23: note = Description of sequence: 23-nt miR-20a
  source 1..23: mol_type = other RNA
  modified_base 1: mod_base = OTHER; note = monophosphate group attached
taaagtgcttatacccccccccc